(19) United States
(12) Patent Application Publication
(10) Pub. No.: US 2022/0411840 A1
(43) Pub. Date: Dec. 29, 2022

(54) High Efficiency Template-Free Enzymatic Synthesis of Polynucleotides

(71) Applicant: DNA Script, Le Kremlin-Bicêtre (FR)

(72) Inventors: Elise Champion, Paris (FR); Mikhael Soskine, Franconville (FR); Elodie Sune, Le Kremlin-Bicêtre (FR); Thomas Ybert, Paris (FR); Xavier Godron, Le Kremlin-Bicêtre (FR); Tillmann Heinisch, Le Kremlin-Bicêtre (FR)

(21) Appl. No.: 17/775,845

(22) PCT Filed: Nov. 9, 2020
(86) PCT No.: PCT/EP2020/081485

§ 371 Date: May 10, 2022

(30) Foreign Application Priority Data
Nov. 13, 2019 (EP) 19208760.9

(51) Int. Cl. C12P 19/34; C12N 9/12

(52) U.S. Cl. CPC C12P 19/34; C12N 9/1264; C12Y 207/07031

(57) ABSTRACT

The invention is directed to compositions and methods for enzymatic template-free synthesis of polynucleotides wherein different terminal deoxynucleotidyl transferase (TdT) variants are used to incorporate different 3′-O-reversibly blocked deoxynucleoside triphosphates (dNTPs) into a growing chain. In part, the invention is a recognition and appreciation that different TdT variants can be engineered to preferentially incorporate specific dNTPs with higher efficiency than a single “general purpose” TdT variant used to incorporate all four 3-O-reversibly blocked dNTPs.

### BACKGROUND

The use of highly purified inexpensive polynucleotides of predetermined sequences in a wide range of lengths has become central to a host of technologies, including genomic and diagnostic sequencing, multiplex nucleic acid amplification, therapeutic antibody development, synthetic biology, nucleic acid-based therapeutics, DNA origami, DNA-based data storage, and the like. Recently, interest has arisen in supplementing or replacing chemically-based synthesis methods by enzymatically-based methods using template-free polymerases, such as, terminal deoxynucleotidyl transferase (TdT), because of the proven efficiency of such enzymes and the benefit of mild non-toxic reaction conditions, e.g. Ybert et al, International patent publication WO2015/159023; Hiatt et al, U.S. Pat. No. 5,763,594; Jensen et al, Biochemistry, 57: 1821-1832 (2018); and the like. Most approaches in enzyme-based synthesis require the use of reversibly blocked nucleoside triphosphates in order to obtain a desired sequence in the polynucleotide product. Unfortunately, natural TdTs incorporate such modified nucleoside triphosphates with reduced efficiency as compared to unmodified nucleoside triphosphates. Thus, a great deal of work has been directed to developing new TdT variants with better incorporation efficiencies for modified nucleoside triphosphates, e.g. Champion et al, U.S. patent publication US2019/0211315; Ybert et al, International patent publication WO2017/216472, and the like.

In view of the above, the field of template-free enzymatically-based polynucleotide synthesis would be advanced if new template-free polymerases, such as new TdT variants, and methods of use, were available for improved incorporation of reversibly blocked nucleoside triphosphates.

### SUMMARY OF THE INVENTION

The present invention is directed to methods, compositions and kits for template-free enzymatic synthesis of polynucleotides, which include terminal deoxynucleotidyl transferase (TdT) variants that display enhanced efficiency in incorporating nucleoside triphosphates under different reaction circumstances, including (i) when different kinds of reversibly blocked nucleoside triphosphates are being incorporated, and (ii) in the presence of different secondary structures (e.g. hairpins, cross-strand hybrids, intra-strand hybrids, G-quartets, and the like) in polynucleotide intermediates of the desired product. More particularly, the invention is in part a recognition and appreciation that different TdT variants may be engineered to incorporate different kinds of 3′-O-blocked nucleoside triphosphates with different efficiencies and that such differences in efficiency can be used to improve overall synthesis efficiency by using in the same process different TdT variants with different kinds of 3′-O-blocked nucleoside triphosphates, or subsets of 3′-O-blocked nucleoside triphosphates. Likewise, the invention is in part a recognition and appreciation that different TdT variants may be engineered to incorporate 3′-O-blocked nucleoside triphosphates in the presence of different secondary structures that may form in polynucleotide intermediates.

In some embodiments, the invention is directed to compositions comprising a terminal deoxynucleotidyl transferase (TdT) variant (an “ACT-TdT variant”) comprising an amino acid sequence at least ninety percent identical to an amino acid of SEQ ID NO: 1 and having the following substitutions with respect to SEQ ID NO: 1: A17V+L52F+G57E+M63R+A108V+K147R+C173G+R207L+M210Q+R325V+E328K+N345E+R351K, wherein the TdT variant (i) is capable of synthesizing a nucleic acid fragment without a template and (ii) is capable of incorporating each of a 3′-O-modified dATP, dCTP or dTTP onto a free 3′-hydroxyl of a nucleic acid fragment at a higher rate than a TdT of reference that displays substantially equal incorporation rates for all dNTPs.

In some embodiments, the invention is directed to compositions comprising a terminal deoxynucleotidyl transferase (TdT) variant (a “G-TdT variant”) comprising an amino acid sequence at least ninety percent identical to an amino acid of SEQ ID NO: 1 and having the following substitutions with respect to SEQ ID NO: 1: A17V+L52F+G57E+M63R+176V+A108V+C173G+R207L+F259E+Q261R+K265G+R325V+E328N+R351K, wherein the TdT variant (i) is capable of synthesizing a nucleic acid fragment without a template and (ii) is capable of incorporating a 3′-O-modified dGTP onto a free 3′-hydroxyl of a nucleic acid fragment at a higher rate than a TdT of reference that displays substantially equal incorporation rates for all dNTPs.

In some embodiments, the TdT variant of SEQ ID NO: 2 (M27) is employed as a TdT of reference to assess and compare rates of incorporation by other TdT variants, such as, for example, ACT-TdT or G-TdT variants.

It is also an object of the present invention to provide a terminal deoxynucleotidyltransferase (TdT) variant comprising an amino acid sequence with at least 80%, 85%, 90%, 95%, 99% or 100% identity to the full length amino acid sequence set forth in SEQ ID NO: 1, except (ii) the combination of substitutions A17V+L52F+G57E+M63R+A108V+K147R+C173G+R207L+M210Q+R325V+E328K+N345E+R351K, as compared to SEQ ID NO:1, (iii) is capable of synthesizing a nucleic acid fragment without a template and (iv) is capable of incorporating a modified nucleotide into a nucleic acid fragment. In a particular embodiment, the TdT comprises the combination of substitutions A17V+Q37E+D41R+L52F+G57E+M63R+S94R+G98E+A108V+S146E+K147R+Q149R+C173G+M184T+R207L+K209H+M210Q+G284L+E289A+R325V+E328K+N345E+R351K, as compared to SEQ ID NO:1.

It is another object of the invention to provide a terminal deoxynucleotidyltransferase (TdT) variant comprising an amino acid sequence with at least 80%, 85% 90%, 95%, 99% or 100% identity to the full length amino acid sequence set forth in SEQ ID NO: 1, (ii) except the combination of substitutions A17V+L52F+G57E+M63R+176V+A108V+C173G+R207L+F259E+Q261R+K265G+R325V+E328N+R351K, as compared to SEQ ID NO: 1, wherein the TdT variant, (iii) is capable of synthesizing a nucleic acid fragment without a template and (iv) is capable of incorporating a modified nucleotide into a nucleic acid fragment. In a particular embodiment, the TdT comprises the combination of substitutions A17V+Q37E+D41R+L52F+G57E+M63R+I76V+S94R+G98E+A108V+S146E+Q149R+C173G+M184T+R207L+K209H+F259E+Q261R+K265G+G284L+E289A+R325V+E328N+R351K, as compared to SEQ ID NO:1.

In one aspect, the invention is directed to methods of using compositions of the above TdT variants, or like TdT variants, to synthesize a polynucleotide comprising the steps of (a) providing an initiator having a free 3′-hydroxyl; and (b) repeating cycles of (i) contacting under elongation conditions the initiator or elongated fragments having free 3′-O-hydroxyls with a 3′-O-blocked nucleoside triphosphate and a terminal deoxynucleotidyl transferase (TdT) variant so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′-hydroxyls, until the polynucleotide is synthesized, wherein a first TdT variant elongates the initiator or elongated fragments with a 3′-O-blocked nucleoside triphosphate of a first set of 3′-O-blocked nucleoside triphosphates and a second TdT variant elongates the initiator or elongated fragments with a 3′-O-blocked nucleoside triphosphate of a second set of 3′-O-blocked nucleoside triphosphates, and wherein the first TdT variant elongates the initiator or elongated fragments with 3′-O-blocked nucleoside triphosphates from the first set with greater efficiency than the second TdT variant and the second TdT variant elongates the initiator or elongated fragments with 3′-O-blocked nucleoside triphosphates from the second set with greater efficiency than the first TdT variant. In some embodiments, an ACT-TdT variant is the first TdT variant and a G-TdT variant is the second TdT variant.

In some embodiments, first, second and third TdT variants may be employed that each have the highest rate of incorporation of 3′-O-blocked nucleoside triphosphates from first, second and third sets thereof, respectively. In still other embodiments, first, second, third and fourth TdT variants may be employed that each have the highest rate of incorporation of 3′-O-blocked nucleoside triphosphates with respect to a first, second, third and fourth 3′-O-blocked-nucleoside triphosphate, respectively.

In some embodiments, first and second; first, second and third; or first, second, third and fourth TdT variants may be used separately with 3′O-blocked dNTPs of their respective sets; in other embodiments, such TdT variants may be used as mixtures in each cycle of incorporation and de-protection.

In some embodiments, the invention is directed to kits for practicing the above methods wherein the kits comprise a first TdT variant and a second TdT variant, and wherein (i) the first TdT variant incorporates 3′-O-modified dNTPs of a first set onto an initiator or an elongated fragment at a higher rate than that of a second TdT variant, (ii) the second TdT variant incorporates 3′-O-modified dNTPs of a second set onto an initiator or an elongated fragment at a higher rate than that of the first TdT variant, and (iii) the first and second sets of dNTPs are nonoverlapping.

In some embodiments, an ACT-TdT variant comprises a TdT that is at least 90 percent identical to the amino acid sequence of SEQ ID NO: 1 and that has the following substitutions: A17V+Q37E+D41R+L52F+G57E+M63R+S94R+G98E+A108V+S146E+K147R+Q149R+C173G+M184T+R207L+K209H+M210Q+G284L+E289A+R325V+E328K+N345E+R351K. Such TdT variants include TdT variant M55, which is 100 percent identical to the amino acid sequence of SEQ ID NO: 1 subject to the substitutions of the foregoing sentence. In further embodiments, an ACT-TdT variant includes SEQ ID NO: 15 (M55-1), SEQ ID NO: 16 (M55-2) with mutation Q4E.

In some embodiments, a G-TdT variant comprises a TdT that is at least 90 percent identical to the amino acid sequence of SEQ ID NO: 1 and that has the following substitutions: A17V+Q37E+D41R+L52F+G57E+M63R+I76V+S94R+G98E+A108V+S146E+Q149R+C173G+M184T+R207L+K209H+F259E+Q261R+K265G+G284L+E289A+R325V+E328N+R351K. Such TdT variants include TdT variant M56, which is 100 percent identical to the amino acid sequence of SEQ ID NO: 1 subject to the substitutions of the foregoing sentence. In further embodiments, a G-TdT variant includes SEQ ID NO: 9 (M33), SEQ ID NO: 10 (M33-1), SEQ ID NO: 11 (M33-2) with mutation Q4E, SEQ ID NO: 13 (M56-1) and SEQ ID NO: 14 (M56-2) with mutation Q4E.

In some embodiments, the above percent identity value is at least 80 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity.

In regard to (ii), such 3′-O-modified nucleotide may comprise a 3′-O-NH2-nucleoside triphosphate, a 3′-O-azidomethyl-nucleoside triphosphate, a 3′-O-allyl-nucleoside triphosphate, a 3′O-(2-nitrobenzyl)-nucleoside triphosphate, or a 3′-O-propargyl-nucleoside triphosphate.

### BRIEF DESCRIPTION OF THE DRAWINGS

FIG. 1 illustrates diagrammatically the steps of a method of template-free enzymatic nucleic acid synthesis using TdT variants of the invention.

### DETAILED DESCRIPTION OF THE INVENTION

While the invention is amenable to various modifications and alternative forms, specifics thereof have been shown by way of example in the drawings and will be described in detail. It should be understood that the intention is not to limit the invention to the particular embodiments described. It is the intention to cover all modifications, equivalents, and alternatives falling within the spirit and scope of the invention. Guidance for aspects of the invention is found in many available references and treatises well known to those with ordinary skill in the art, including, for example, Sambrook et al. (1989), Molecular cloning: A Laboratory Manual, Cold Spring Harbor Laboratory, and the like.

The invention is directed to methods and kits for enzymatically synthesizing polynucleotides having predetermined sequences using different TdT variants having enhanced dNTP incorporation efficiencies under different reaction circumstances in order to enhance overall synthesis efficiency. In some embodiments, different TdT variants are used separately in different synthesis cycle steps that present reaction circumstances (e.g. type of 3′-O-blocked dNTP being incorporated) for which such TdT variant provides maximum incorporation efficiency. In other embodiments, different TdT variants are used as a mixture so that a TdT variant is always present in a reaction mixture that provides maximum incorporation efficiency but which must also compete with other TdT variants present for implementing a coupling reaction. In some embodiments of the latter type, a ratio of TdT variants in a mixture is selected to minimize polynucleotide synthesis time for a given or an expected nucleotide composition of the target polynucleotides to be synthesized. In some embodiments, such given nucleotide composition is A:C:G:T=1:1:1:1.

In some embodiments, methods of the invention are carried out with the following steps: a) providing an initiator having a free 3′-hydroxyl; and b) repeating cycles of (i) contacting under elongation conditions the initiator or elongated fragments having free 3′-O-hydroxyls with a 3′-O-blocked nucleoside triphosphate and a terminal deoxynucleotidyltransferase (TdT) variant so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is synthesized, wherein a first TdT variant elongates the initiator or elongated fragments with a 3′-O-blocked nucleoside triphosphate selected from a first set of 3′-O-blocked nucleoside triphosphates and a second TdT variant, different from the first TdT variant, elongates the initiator or elongated fragments with a 3′-O-blocked nucleoside triphosphate selected from a second set of 3′-O-blocked nucleoside triphosphates, and wherein the first TdT variant elongates the initiator or elongated fragments with 3′-O-blocked nucleoside triphosphates from the first set with greater efficiency than the second TdT variant and the second TdT variant elongates the initiator or elongated fragments with 3′-O-blocked nucleoside triphosphates from the second set with greater efficiency than the first TdT variant.

In other embodiments, methods of the invention are carried out with the following steps: a) providing an initiator having a free 3′-hydroxyl; and b) repeating cycles of (i) contacting under elongation conditions the initiator or elongated fragments having free 3′-O-hydroxyls with a 3′-O-blocked nucleoside triphosphate and a TdT mixture so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is synthesized, wherein the TdT mixture comprises a first TdT variant that elongates the initiator or elongated fragments with a 3′-O-blocked nucleoside triphosphate selected from a first set of 3′-O-blocked nucleoside triphosphates and a second TdT variant, different from the first TdT variant, that elongates the initiator or elongated fragments with a 3′-O-blocked nucleoside triphosphate selected from a second set of 3′-O-blocked nucleoside triphosphates, and wherein the first TdT variant elongates the initiator or elongated fragments with 3′-O-blocked nucleoside triphosphates from the first set with greater efficiency than the second TdT variant and the second TdT variant elongates the initiator or elongated fragments with 3′-O-blocked nucleoside triphosphates from the second set with greater efficiency than the first TdT variant. In some embodiments, the TdT mixture comprises the first TdT variant and the second TdT variant in a ratio that minimizes a time for synthesizing the desired polynucleotide at a given product yield.

### TdT Variants

TdT variants of the invention as described above each comprise an amino acid sequence having a percent sequence identity with a specified SEQ ID NO, subject to the presence of indicated substitutions. In some embodiments, the number and type of sequence differences between a TdT variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 1A.

TABLE 1A
Synonymous Sets of Amino Acids I

| Amino Acid | Synonymous Set |
|---|---|
| Ser | Ser, Thr, Gly, Asn |
| Arg | Arg, Gin, Lys, Glu, His |
| Leu | He, Phe, Tyr, Met, Val, Leu |
| Pro | Gly, Ala, Thr, Pro |
| Thr | Pro, Ser, Ala, Gly, His, Gin, Thr |
| Ala | Gly, Thr, Pro, Ala |
| Val | Met, Tyr, Phe, He, Leu, Val |
| Gly | Gly, Ala, Thr, Pro, Ser |
| He | Met, Tyr, Phe, Val, Leu, He |
| Phe | Trp, Met, Tyr, He, Val, Leu, Phe |
| Tyr | Trp, Met, Phe, He, Val, Leu, Tyr |
| Cys | Cys, Ser, Thr |
| His | His, Glu, Lys, Gin, Thr, Arg |
| Gin | Gin, Glu, Lys, Asn, His, Thr, Arg |
| Asn | Asn, Gin, Asp, Ser |
| Lys | Lys, Glu, Gin, His, Arg |
| Asp | Asp, Glu, Asn |
| Glu | Glu, Asp, Lys, Asn, Gin, His, Arg |
| Met | Met, Phe, He, Val, Leu |
| Trp | Trp |

In some embodiments, sets of synonymous amino acids that may be employed are set forth in Table 1B.

TABLE IB
Synonymous Sets of Amino Acids II

| Amino Acid | Synonymous Set |
|---|---|
| Ser | Ser |
| Arg | Arg, Lys, His |
| Leu | He, Phe, Met, Leu |
| Pro | Ala, Pro |
| Thr | Thr |
| Ala | Pro, Ala |
| Val | Met, He Val |
| Gly | Gly |
| He | Met, Phe, Val, Leu, He |
| Phe | Met, Tyr, He, Leu, Phe |
| Tyr | Trp, Met |
| Cys | Cys, Ser |
| His | His, Gin, Arg |
| Gin | Gin, Glu, His |
| Asn | Asn, Asp |
| Lys | Lys, Arg |
| Asp | Asp, Asn |
| Glu | Glu, Gin |
| Met | Met, Phe, He, Val, Leu |
| Trp | Trp |

### Measurement of Nucleotide Incorporation Activity

The efficiency of nucleotide incorporation by variants of the invention may be measured by an extension, or elongation, assay, e.g. as described in Boule et al (cited below); Bentolila et al (cited below); and Hiatt et al, U.S. Pat. No. 5,808,045, the latter of which is incorporated herein by reference. Briefly, in one form of such an assay, a fluorescently labeled oligonucleotide having a free 3′-hydroxyl is reacted under TdT extension conditions with a variant TdT to be tested for a predetermined duration in the presence of a reversibly blocked nucleoside triphosphate, after which the extension reaction is stopped and the amounts of extension products and unextended initiator oligonucleotide are quantified after separation by gel electrophoresis. By such assays, the incorporation efficiency of a variant TdT may be readily compared to the efficiencies of other variants or to that of wild type or reference TdTs, or other polymerases. In some embodiments, a measure of variant TdT efficiency may be a ratio (given as a percentage) of amount of extended product using the variant TdT over the amount of extended product using wild type TdT in an equivalent assay.

In some embodiments, the following particular extension assay may be used to measure incorporation efficiencies of TdTs: Primer used is the following:

(SEQ ID NO: 3)
5′-AAAAAAAAAAAAAAGGGG-3′

The primer has also an ATTO fluorescent dye on the 5′ extremity. Representative modified nucleotides used (noted as dNTP in Table 2) include 3′-O-amino-2′,3′-dideoxynucleotides-5′-triphosphates (ONH2, Firebird Biosciences), such as 3′-O-amino-2′,3′-dideoxyadenosine-5′-triphosphate. For each different variant tested, one tube is used for the reaction. The reagents are added in the tube, starting from water, and then in the order of Table 2. After 30 min at 37° C. the reaction is stopped by addition of formamide (Sigma).

TABLE 2
Extension Activity Assay Reagents

| Reagent | Concentration | Volume |
|---|---|---|
| H₂O | — | 12 μL |
| Activity buffer | 10x | 2 μL |
| dNTP | 250 μM | 2 μL |
| Purified enzyme | 20 μM | 2 μL |
| Fluorescent primer | 500 μM | 2 μL |

The Activity buffer comprises, for example, TdT reaction buffer (available from New England Biolabs) supplemented with CoCl₂.

The product of the assay is analyzed by conventional polyacrylamide gel electrophoresis. For example, products of the above assay may be analyzed in a 16 percent polyacrylamide denaturing gel (Bio-Rad). Gels are made just before the analysis by pouring polyacrylamide inside glass plates and let it polymerize. The gel inside the glass plates is mounted on an adapted tank filed with TBE buffer (Sigma) for the electrophoresis step. The samples to be analyzed are loaded on the top of the gel. A voltage of 500 to 2,000V is applied between the top and bottom of the gel for 3 to 6 h at room temperature. After separation, gel fluorescence is scanned using, for example, a Typhoon scanner (GE Life Sciences). The gel image is analyzed using ImageJ software (imagej.nih.gov/ij/), or its equivalent, to calculate the percentage of incorporation of the modified nucleotides.

Hairpin completion assay. In one aspect, the invention includes methods of measuring the capability of a polymerase, such as a TdT variant, to incorporate a dNTP onto a 3′ end of a polynucleotide (i.e. a “test polynucleotide”). One such method comprises providing a test polynucleotide with a free 3′ hydroxyl under reaction conditions in which it is substantially only single stranded, but that upon extension with a polymerase, such as a TdT variant, forms a stable hairpin structure comprising a single stranded loop and a double stranded stem, thereby allowing detection of an extension of the 3′ end by the presence of the double stranded polynucleotide. The double stranded structure may be detected in a variety of ways including, but not limited to, fluorescent dyes that preferentially fluoresce upon intercalation into the double stranded structure, fluorescent resonance energy transfer (FRET) between an acceptor (or donor) on the extended polynucleotide and a donor (or acceptor) on an oligonucleotide that forms a triplex with the newly formed hairpin stem, FRET acceptors and donors that are both attached to the test polynucleotide and that are brought into FRET proximity upon formation of a hairpin, or the like. In some embodiments, a stem portion of a test polynucleotide after extension by a single nucleotide is in the range of 4 to 6 basepairs in length; in other embodiments, such stem portion is 4 to 5 basepairs in length; and in still other embodiments, such stem portion is 4 basepairs in length. In some embodiments, a test polynucleotide has a length in the range of from 10 to 20 nucleotides; in other embodiments, a test polynucleotide has a length in the range of from 12 to 15 nucleotides. In some embodiments, it is advantageous or convenient to extend the test polynucleotide with a nucleotide that maximizes the difference between the melting temperatures of the stem without extension and the stem with extension; thus, in some embodiments, a test polynucleotide is extended with a dC or dG (and accordingly the test polynucleotide is selected to have an appropriate complementary nucleotide for stem formation).

Exemplary test polynucleotides for hairpin completion assays include p875 (5′-CAGTTAAAAACT) (SEQ ID NO: 4) which is completed by extending with a dGTP; p876 (5′-GAGTTAAAACT) (SEQ ID NO: 5) which is completed by extending with a dCTP; and p877 (5′-CAGCAAGGCT) (SEQ ID NO: 6) which is completed by extending with a dGTP. Exemplary reaction conditions for such test polynucleotides may comprise: 2.5-5 μM of test polynucleotide, 1:4000 dilution of GelRed® (intercalating dye from Biotium, Inc., Fremont, Calif.), 200 mM Cacodylate KOH pH 6.8, 1 mM CoCl₂, 0-20% of DMSO and 3′ ONH₂ dGTP and TdT at desired concentrations. Completion of the hairpin may be monitored by an increase in fluorescence of GelRed® dye using a conventional fluorimeter, such as a TECAN reader at a reaction temperature of 28-38° C., using an excitation filter set to 360 nm and an emission filter set to 635 nm.

In some embodiments of this aspect of the invention, TdT variants may be tested for their capacity for template-free incorporate of nucleoside triphosphates by the following steps: (a) combining a test polynucleotide having a free 3′-hydroxyl, a TdT variant and a nucleoside triphosphate under conditions wherein the test polynucleotide is single stranded but upon incorporation of the nucleoside triphosphate forms a hairpin having a double stranded stem region, and (b) detecting the amount of double stranded stem regions formed as a measure of the capacity of the TdT variant to incorporate the nucleoside triphosphate. In some embodiments, the nucleoside triphosphate is a 3′-O-blocked nucleoside triphosphate.

### Template-Free Enzymatic Synthesis

Template-free enzymatic synthesis of polynucleotides may be carried out by a variety of known protocols using template-free polymerases, such as terminal deoxynucleotidyl transferase (TdT), including variants thereof engineered to have improved characteristics, such as greater temperature stability or greater efficiency in the incorporation of 3′-O-blocked deoxynucleoside triphosphates (3′-O-blocked dNTPs), e.g. Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

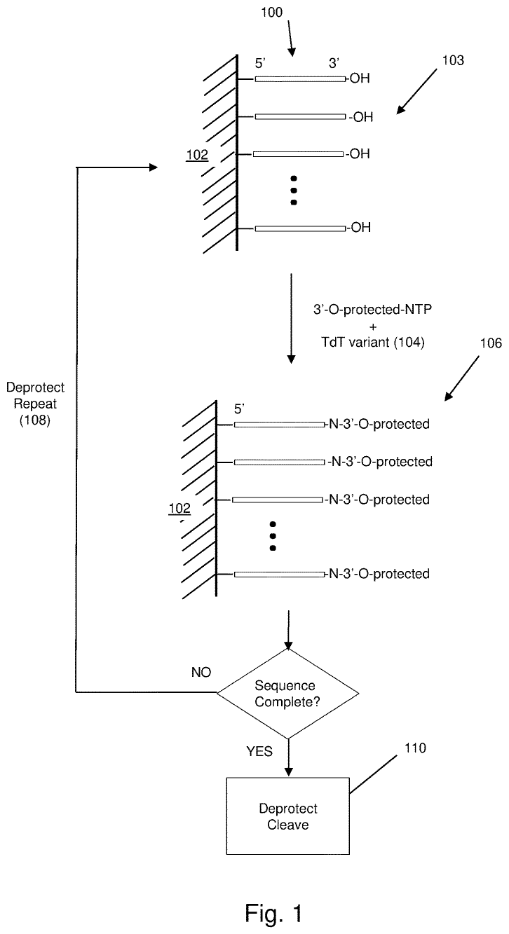

In some embodiments, the method of enzymatic DNA synthesis comprises repeated cycles of steps, such as are illustrated in FIG. 1, in which a predetermined nucleotide is added in each cycle. Initiator polynucleotides (100) are provided, for example, attached to solid support (102), which have free 3′-hydroxyl groups (103). To the initiator polynucleotides (100) (or elongated initiator polynucleotides in subsequent cycles) are added a 3′-O-protected-dNTP and a TdT variant under conditions (104) effective for the enzymatic incorporation of the 3′-O-protected-dNTP onto the 3′ end of the initiator polynucleotides (100) (or elongated initiator polynucleotides). This reaction produces elongated initiator polynucleotides whose 3′-hydroxyls are protected (106). If the elongated initiator polynucleotide contains a competed sequence, then the 3′-O-protection group is removed, or deprotected, and the desired sequence is cleaved from the original initiator polynucleotide. Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

In some embodiments, an ordered sequence of nucleotides are coupled to an initiator nucleic acid using a TdT in the presence of 3′-O-reversibly blocked dNTPs in each synthesis step. In some embodiments, the method of synthesizing an oligonucleotide comprises the steps of (a) providing an initiator having a free 3′-hydroxyl; (b) reacting under extension conditions the initiator or an extension intermediate having a free 3′-hydroxyl with a TdT in the presence of a 3′-O-blocked nucleoside triphosphate to produce a 3′-O-blocked extension intermediate; (c) deblocking the extension intermediate to produce an extension intermediate with a free 3′-hydroxyl; and (d) repeating steps (b) and (c) until the polynucleotide is synthesized. (Sometime “an extension intermediate” is also referred to as an “elongation fragment.”) In some embodiments, an initiator is provided as an oligonucleotide attached to a solid support, e.g. by its 5′ end. The above method may also include washing steps after the reaction, or extension, step, as well as after the de-blocking step. For example, the step of reacting may include a sub-step of removing unincorporated nucleoside triphosphates, e.g. by washing, after a predetermined incubation period, or reaction time. Such predetermined incubation periods or reaction times may be a few seconds, e.g. 30 sec, to several minutes, e.g. 30 min.

The above method may also include capping step(s) as well as washing steps after the reacting, or extending, step, as well as after the deblocking step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions.

In some embodiments that comprise serial synthesis of oligonucleotides, capping steps may be undesirable as capping may prevent the production of equal molar amounts of a plurality of oligonucleotides. Without capping, sequences will have a uniform distribution of deletion errors, but each of a plurality of oligonucleotides will be present in equal molar amounts. This would not be the case where non-extended fragments are capped.

In some embodiments, reaction conditions for an extension or elongation step may comprising the following: 2.0 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes. In embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out in a 50 μL volume, at a temperature within the range of RT to 45° C. for 30 seconds to several minutes.

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: U.S. Pat. Nos. 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments, such as those described above. It is understood that other blocking group pairs, or groups containing more than two, may be available for use in these embodiments of the invention.

| | |
|---|---|
| 3′—O—NH2 | 3′—O-azidomethyl |
| 3′—O—NH2 | 3′—O-allyl |
| 3′—O—NH2 | 3′—O-phosphate |
| 3′—O-azidomethyl | 3′—O-allyl |
| 3′—O-azidomethyl | 3′—O-phosphate |
| 3′—O-allyl | 3′—O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C.

A “3′-phosphate-blocked” or “3′-phosphate-protected” nucleotide refers to nucleotides in which the hydroxyl group at the 3′-position is blocked by the presence of a phosphate containing moiety. Examples of 3′-phosphate-blocked nucleotides in accordance with the invention are nucleotidyl-3′-phosphate monoester/nucleotidyl-2′,3′-cyclic phosphate, nuclcotidyl-2′-phosphate monoester and nucleotidyl-2′ or 3′-alkylphosphate diester, and nucleotidyl-2′ or 3′-pyrophosphate. Thiophosphate or other analogs of such compounds can also be used, provided that the substitution does not prevent dephosphorylation resulting in a free 3′-OH by a phosphatase.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999); Ferrero et al, Monatshefte fur Chemie, 131: 585-616 (2000); Taunton-Rigby et al, J. Org. Chem., 38(5): 977-985 (1973); Uemura et al, Tetrahedron Lett., 30(29): 3819-3820 (1989); Becker et al, J. Biol. Chem., 242(5): 936-950 (1967); Tsien, International patent publication WO1991/006678.

As used herein, an “initiator” (or equivalent terms, such as, “initiating fragment,” “initiator nucleic acid,” “initiator oligonucleotide,” or the like) refers to a short oligonucleotide sequence with a free 3′-end, which can be further elongated by a template-free polymerase, such as TdT. In one embodiment, the initiating fragment is a DNA initiating fragment. In an alternative embodiment, the initiating fragment is an RNA initiating fragment. In one embodiment, the initiating fragment possesses between 3 and 100 nucleotides, in particular between 3 and 20 nucleotides. In one embodiment, the initiating fragment is single-stranded. In an alternative embodiment, the initiating fragment is double-stranded. In a particular embodiment, an initiator oligonucleotide synthesized with a 5′-primary amine may be covalently linked to magnetic beads using the manufacturer's protocol. Likewise, an initiator oligonucleotide synthesized with a 3′-primary amine may be covalently linked to magnetic beads using the manufacturer's protocol. A variety of other attachment chemistries amenable for use with embodiments of the invention are well-known in the art, e.g. Integrated DNA Technologies brochure, “Strategies for Attaching Oligonucleotides to Solid Supports,” v. 6 (2014); Hermanson, Bioconjugate Techniques, Second Edition (Academic Press, 2008); and like references.

Many of the 3′-O-blocked dNTPs employed in the invention may be purchased from commercial vendors or synthesized using published techniques, e.g. U.S. Pat. No. 7,057,026; International patent publications WO2004/005667, WO91/06678; Canard et al, Gene (cited above); Metzker et al, Nucleic Acids Research, 22: 4259-4267 (1994); Meng et al, J. Org. Chem., 14: 3248-3252 (3006); U.S. patent publication 2005/037991. In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

—O—Z

wherein —Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula=C(R′″)₂ wherein each R′″ may be the same or different and is selected from the group comprising hydrogen and halogen atoms and alkyl groups, with the proviso that in some embodiments the alkyl of each R′″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In certain embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, —Z of the modified nucleotide or nucleoside is of formula —C(R′)₂—N3. In certain embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, Mass.).

In a further particular embodiments, the 3′-blocked nucleotide triphosphate is blocked by either a 3′-O-azidomethyl, 3′-O—NH₂ or 3′-O-allyl group.

In still other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

### Production of Variant TdTs

Variants of the invention may be produced by mutating known reference or wild type TdT-coding polynucleotides, then expressing it using conventional molecular biology techniques. For example, the mouse TdT gene (SEQ ID NO: 1) may be assembled from synthetic fragments using conventional molecular biology techniques, e.g. using protocols described by Stemmer et al, Gene, 164: 49-53 (1995); Kodumal et al, Proc. Natl. Acad. Sci., 101: 15573-15578 (2004); or the like, or it may be directly cloned from mouse cells using protocols described by Boule et al, Mol. Biotechnology, 10: 199-208 (1998), or Bentolila et al, EMBO J., 14: 4221-4229 (1995); or the like.

For example, an isolated TdT gene may be inserted into an expression vector, such as pET32 (Novagen) to give a vector pCTdT which then may be used to make and express variant TdT proteins using conventional protocols. Vectors with the correct sequence may be transformed in E. coli producer strains.

Transformed strains are cultured using conventional techniques to pellets from which TdT protein is extracted. For example, previously prepared pellets are thawed in 30 to 37° C. water bath. Once fully thawed, pellets are resuspended in lysis buffer composed of 50 mM tris-HCL (Sigma) pH 7.5, 150 mM NaCl (Sigma), 0.5 mM mercaptoethanol (Sigma), 5% glycerol (Sigma), 20 mM imidazole (Sigma) and 1 tab for 100 mL of protease cocktail inhibitor (Thermofisher). Careful resuspension is carried out in order to avoid premature lysis and remaining of aggregates. Resuspended cells are lysed through several cycles of French press, until full color homogeneity is obtained. Usual pressure used is 14,000 psi. Lysate is then centrifuged for 1 h to 1 h30 at 10,000 rpm. Centrifugate is pass through a 0.2 μm filter to remove any debris before column purification.

TdT protein may be purified from the centrifugate in a one-step affinity procedure. For example, Ni-NTA affinity column (GE Healthcare) is used to bind the polymerases. Initially the column has been washed and equilibrated with 15 column volumes of 50 mM tris-HCL (Sigma) pH 7.5, 150 mM NaCl (Sigma) and 20 mM imidazole (Sigma). Polymerases are bound to the column after equilibration. Then a washing buffer, composed of 50 mM tris-HCL (Sigma) pH 7.5, 500 mM NaCl (Sigma) and 20 mM imidazole (Sigma), is applied to the column for 15 column volumes. After wash the polymerases are eluted with 50 mM tris-HCL (Sigma) pH 7.5, 500 mM NaCl (Sigma) and 0.5M imidazole (Sigma). Fractions corresponding to the highest concentration of polymerases of interest are collected and pooled in a single sample. The pooled fractions are dialyzed against the dialysis buffer (20 mM Tris-HCl, pH 6.8, 200 mM Na Cl, 50 mM MgOAc, 100 mM [NH4]2SO4). The dialysate is subsequently concentrated with the help of concentration filters (Amicon Ultra-30, Merk Millipore). Concentrated enzyme is distributed in small aliquots, 50% glycerol final is added, and those aliquots are then frozen at −20° C. and stored for long term. 5 μL of various fraction of the purified enzymes are analyzed in SDSPAGE gels.

In some embodiments, a TdT variant may be operably linked to a linker moiety including a covalent or non-covalent bond; amino acid tag (e.g., poly-amino acid tag, poly-His tag, 6His-tag, or the like); chemical compound (e.g., polyethylene glycol); protein-protein binding pair (e.g., biotin-avidin); affinity coupling; capture probes; or any combination of these. The linker moiety can be separate from or part of a TdT variant. An exemplary His-tag for use with TdT variants of the invention is MASSHHHHHHSSGSENLYFQTGSSG- (SEQ ID NO: 12)). The tag-linker moiety does not interfere with the nucleotide binding activity, or catalytic activity of the TdT variant.

The above processes, or equivalent processes, result in an isolated TdT variant that may be mixed with a variety of reagents, such as, salts, pH buffers, and the like, that are necessary or useful for activity and/or preservation, thereby providing a formulation that can be used in methods of the invention.

### Kits for Practicing Methods of the Invention

The invention includes a variety of kits for practicing methods of the invention. In one aspect, kits of the invention comprise a plurality of TdT variants of the invention in a formulation, or in formulations, suitable for carrying out template-free enzymatic polynucleotide synthesis as described herein. In some embodiments, the plurality of TdT variants is between 2 and 4. In other embodiments, the plurality is 2. Such kits may also include synthesis buffers for each TdT variant that provide reaction conditions for optimizing the template-free addition or incorporation of a 3′-O-protected dNTP to a growing strand. In some embodiments, each of the plurality of TdT variants may be provided separately, in the same or different formulations, in separate containers, and in other embodiments, some or all of the TdT variants may be provided in mixtures thereof in a common formulation. In some embodiments, kits of the invention further include 3′-O-reversibly protected dNTPs. In such embodiments, the 3′-O-reversibly protected dNTPs may comprise 3′-O-amino-dNTPs or 3′—O-azidomethyl-dNTPs. In further embodiments, kits may include one or more of the following items, either separately or together with the above-mentioned items: (i) deprotection reagents for carrying out a deprotecting step as described herein, (ii) solid supports with initiators attached thereto, (iii) cleavage reagents for releasing completed polynucleotides from solid supports, (iv) wash reagents or buffers for removing unreacted 3′-O-protected dNTPs at the end of an enzymatic addition or coupling step, and (v) post-synthesis processing reagents, such as purification columns, desalting reagents, eluting reagents, and the like.

In regard to items (ii) and (iii) above, certain initiators and cleavage reagents go together. For example, an initiator comprising an inosine cleavable nucleotide may come with an endonuclease V cleavage reagent; an initiator comprising a nitrobenzyl photocleavable linker may come with a suitable light source for cleaving the photocleavable linker; an initiator comprising a uracil may come with a uracil DNA glycosylase cleavage reagent; and the like.

### Example 1

### Incorporation Efficiencies of TdT Variants M55 and M56

TdT variants M55 and M56 (defined above) were tested for their ability to incorporate 3′-O-azidomethyl nucleoside triphosphates (AM-dNTPs) on two different primers (synGG: 5′-TGTGAGAGTGAAATGAGG (SEQ ID NO: 7) and polyT: 5′-TTTTTTTTTTTTTTTTTTTT (20T) (SEQ ID NO: 8) in solution under the following reaction conditions: 1 uM primer, 2 uM purified TdT variant, 80 uM AM-dNTP, 10% DMSO, Buffer (100 mM potassium cacodylate, 1 mM CoCl₂, pH: 6.8); Reaction performed for 5 min at 37° C.; Reaction stopped by heating at 95° C. for 5 min. Table 3 shows the percentage of extended primers for the different primers, dNTPs and TdT variant.

TABLE 3
Percentage of AM-dNTP Incorporation

| Primer: | synGG | | | | polyT | | | |
|---|---|---|---|---|---|---|---|---|
| AM-dNTP: | A | T | C | G | A | T | C | G |
| M55 | 74 | 76 | 86 | 30 | 15 | 61 | 69 | 12 |
| M56 | 4 | 71 | 68 | 65 | 0 | 52 | 27 | 15 |

### Definitions

Amino acids are represented by either their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to sequence motifs that are conserved among the amino acid sequences of TdTs of evolutionarily related species, e.g. related by genus, families, or the like. Examples of such conserved sequence motifs are described in Motea et al, Biochim. Biophys. Acta. 1804(5): 1151-1166 (2010); Delarue et al, EMBO J., 21: 427-439 (2002); and like references.

“Isolated” in reference to protein means such a compound which has been identified and separated and/or recovered from a component of its natural environment or from a heterogeneous reaction mixture. Contaminant components of a natural environment or reaction mixture are materials which would interfere with a protein's function, and may include enzymes, hormones, and other proteinaceous or nonproteinaceous solutes. In some embodiments, a protein of the invention is purified (1) to greater than 95% by weight of protein as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. When manufactured by recombinant methodologies, an isolated protein of the invention may include the protein of the invention in situ within recombinant cells since at least one component of the protein's natural environment will not be present. Ordinarily, an isolated protein of the invention is prepared by at least one purification step.

“Kit” refers to any delivery system for delivering materials or reagents for carrying out a method of the invention. In the context of reaction assays, such delivery systems include systems and/or compounds (such as dilutants, surfactants, carriers, or the like) that allow for the storage, transport, or delivery of reaction reagents (e.g., one or more TdT variants, reaction buffers, 3′-O-protected-dNTPs, deprotection reagents, solid suppprts with initiators attached, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain one or more TdT variants for use in a synthesis method, while a second or additional containers may contain deprotection agents, solid supports with initiators, 3′-O-protected dNTPs, or the like.

“Mutant” or “variant,” which are used interchangeably, refer to polypeptides derived from SEQ ID NO: 1, or other specified amino acid sequence, and comprising a modification or an alteration, i.e., a substitution, insertion, and/or deletion, at one or more positions. Such mutants or variant usually have both a template-free polymerase activity and ability to incorporate one or more reversibly blocked nucleoside triphosphate precursors. The variants may be obtained by various techniques well known in the art. In particular, examples of techniques for altering the DNA sequence encoding a wild-type protein, include, but are not limited to, site-directed mutagenesis, random mutagenesis and synthetic oligonucleotide construction, and the like. Mutagenesis activities consist in deleting, inserting or substituting one or several amino-acids in the sequence of a protein or in the case of the invention of a polymerase.

The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

A “substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions.

In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1. A method of synthesizing a polynucleotide having a predetermined sequence, the method comprising the steps of:
a) providing an initiator having a free 3′-hydroxyl; and
b) repeating cycles of (i) contacting under elongation conditions the initiator or elongated fragments having free 3′-O-hydroxyls with a 3′-O-blocked nucleoside triphosphate and a terminal deoxynucleotidyltransferase (TdT) variant so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide is synthesized, wherein a first TdT variant elongates the initiator or elongated fragments with a 3′-O-blocked nucleoside triphosphate selected from a first set of 3′-O-blocked nucleoside triphosphates and a second TdT variant, different from the first TdT variant, elongates the initiator or elongated fragments with a 3′-O-blocked nucleoside triphosphate selected from a second set of 3′-O-blocked nucleoside triphosphates, and wherein the first TdT variant elongates the initiator or elongated fragments with 3′-O-blocked nucleoside triphosphates from the first set with greater efficiency than the second TdT variant and the second TdT variant elongates the initiator or elongated fragments with 3′-O-blocked nucleoside triphosphates from the second set with greater efficiency than the first TdT variant.

2. The method of claim 1, wherein a third TdT variant, different from the first TdT variant and from the second TdT variant, elongates the initiator or elongated fragments with a 3′-O-blocked nucleoside triphosphate selected from a third set of 3′-O-blocked nucleoside triphosphates, and wherein the first TdT variant elongates the initiator or elongated fragments with 3′-O-blocked nucleoside triphosphates from the first set with greater efficiency than the second TdT variant or the third TdT variant, the second TdT variant elongates the initiator or elongated fragments with 3′-O-blocked nucleoside triphosphates from the second set with greater efficiency than the first TdT variant or the third TdT variant, and the third TdT variant elongates the initiator or elongated fragments with 3′-O-blocked nucleoside triphosphates from the third set with greater efficiency than the first TdT variant or the second TdT variant.

3. The method of claim 1 or 2, wherein said first set comprises 3′-O-blocked deoxyadenosine triphosphate, 3′-O-blocked deoxycytidine triphosphate, and 3′-O-blocked deoxythymidine triphosphate and said first TdT variant comprises an amino acid sequence that has at least 90% identity with the amino acid sequence of SEQ ID NO: 1 and comprises the following combination of substitutions: A17V+L52F+G57E+M63R+A108V+K147R+C173G+R207L+M210Q++R325V+E328K+N345E+R351K.

4. The method of claim 3 wherein said first TdT variant comprises the following combination of substitutions: A17V+Q37E+D41R+L52F+G57E+M63R+S94R+G98E+A108V+S146E+K147R+Q149R+C173G+M184T+R207L+K209H+M210Q+G284L+E289A+R325V+E328K+N345E+R351K.

5. The method of anyone of claims 1 to 4, wherein said second set comprises 3′-O-blocked deoxyguanosine triphosphate and said second TdT variant comprises an amino acid sequence that has at least 90% identity to the amino acid sequence of SEQ ID NO: 1 and comprises the following combination of substitutions: A17V+L52F+G57E+M63R+I76V+A108V+C173G+R207L+F259E+Q261R+K265G+R325V+E328N+R351K.

6. The method of claim 5 wherein said second TdT variant has the following combination of substitutions: A17V+Q37E+D41R+L52F+G57E+M63R+I76V+S94R+G98E+A108V+S146E+Q149R+C173G+M184T+R207L+K209H+F259E+Q261R+K265G+G284L+E289A+R325V+E328N+R351K.

7. A terminal deoxynucleotidyltransferase (TdT) comprising an amino acid sequence with at least 80%, 85%, 90%, 95%, 99% identity to the full length amino acid sequence set forth in SEQ ID NO: 1, and (ii) has the combination of substitutions A17V+L52F+G57E+M63R+A108V+K147R+C173G+R207L+M210Q+R325V+E328K+N345E+R351K, as compared to SEQ ID No 1.

8. The TdT according to claim 7, comprising the combination of substitutions A17V+Q37E+D41R+L52F+G57E+M63R+S94R+G98E+A108V+S146E+K147R+Q149R+C173G+M184T+R207L+K209H+M210Q+G284L+E289A+R325V+E328K+N345E+R351K, as compared to SEQ ID NO: 1.

9. A terminal deoxynucleotidyltransferase (TdT) comprising an amino acid sequence with at least 80%, 85%, 90%, 95%, 99% identity to the full length amino acid sequence set forth in SEQ ID NO: 1, and (ii) has the combination of substitutions A17V+L52F+G57E+M63R+176V+A108V+C173G+R207L+F259E+Q261R+K265G+R325V+E328N+R351K, as compared to SEQ ID NO: 1.

10. The TdT according to claim 9, comprising the combination of substitutions A17V+Q37E+D41R+L52F+G57E+M63R+I76V+S94R+G98E+A108V+S146E+Q149R+C173G+M184T+R207L+K209H+F259E+Q261R+K265G+G284L+E289A+R325V+E328N+R351K, as compared to SEQ ID NO:1.

11. Kit for practicing a method of synthesizing a polynucleotide having a predetermined sequence, wherein the kit comprises
a first TdT variant according to claim 7 or 8,
a second TdT variant, according to claim 9 or 10 and
the first and second sets of 3′-O-blocked nucleoside triphosphates, wherein said dNTPs are nonoverlapping.

12. Kit according to claim 11, wherein the first set of dNTPs comprises 3′-O-blocked deoxyadenosine triphosphate, 3′-O-blocked deoxycytidine triphosphate, and 3′-O-blocked deoxythymidine triphosphate and the second set comprises 3′-O-blocked deoxyguanosine triphosphate.


SEQUENCE LISTING (16 sequences)

SEQ ID NO: 1 - protein, 381 aa, ARTIFICIAL SEQUENCE
  truncated mouse TdT
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 381 aa, ARTIFICIAL SEQUENCE
  TdT variant M27
NSSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 3 - DNA, 18 bp, ARTIFICIAL SEQUENCE
  primer sequence for assaying incorporation rate of TdT
aaaaaaaaaaaaaagggg

SEQ ID NO: 4 - DNA, 12 bp, ARTIFICIAL SEQUENCE
  primer for hairpin extension assay to measure incorporation rate of TdT
cagttaaaaact

SEQ ID NO: 5 - DNA, 11 bp, ARTIFICIAL SEQUENCE
  primer for hairpin extension assay to measure incorporation rate of TdT
gagttaaaact

SEQ ID NO: 6 - DNA, 10 bp, ARTIFICIAL SEQUENCE
  primer for hairpin extension assay to measure incorporation rate of TdT
cagcaaggct

SEQ ID NO: 7 - DNA, 18 bp, ARTIFICIAL SEQUENCE
  primer for measuring incorporation rate of TdT
tgtgagagtgaaatgagg

SEQ ID NO: 8 - DNA, 20 bp, ARTIFICIAL SEQUENCE
  primer for measuring incorporation rate of TdT
tttttttttttttttttttt

SEQ ID NO: 9 - protein, 404 aa, Artificial Sequence
  TdT variant M33
MASSHHHHHHSSGSENLYFQSGSSGSPSPVPGSQNVPAPAVKKISQYACQRRTTLNNYNQLFTDALDILAENDEFRGNEGSCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSTLVKEAVVTFLPDALVTMTGGFRLGKMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFNRDLRRYATHERKMMLDNHALYDKTKRVFLEAESEEEIFAHLGLDYIEPWERNA

SEQ ID NO: 10 - protein, 379 aa, Artificial Sequence
  M33-1=TdT variant M33 without affinity tag
SPSPVPGSQNVPAPAVKKISQYACQRRTTLNNYNQLFTDALDILAENDEFRGNEGSCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSTLVKEAVVTFLPDALVTMTGGFRLGKMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFNRDLRRYATHERKMMLDNHALYDKTKRVFLEAESEEEIFAHLGLDYIEPWERNA

SEQ ID NO: 11 - protein, 362 aa, Artificial Sequence
  M33-2=TdT variant M33 without affinity tag and with N-terminal truncation
KISQYACQRRTTLNNYNQLFTDALDILAENDEFRGNEGSCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSTLVKEAVVTFLPDALVTMTGGFRLGKMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFNRDLRRYATHERKMMLDNHALYDKTKRVFLEAESEEEIFAHLGLDYIEPWERNA

SEQ ID NO: 12 - protein, 25 aa, Artificial Sequence
  exemplary affinity tag
MASSHHHHHHSSGSENLYFQTGSSG

SEQ ID NO: 13 - protein, 379 aa, Artificial Sequence
  M56-1=M56 without affinity tag
SPSPVPGSQNVPAPVVKKISQYACQRRTTLNNYNELFTRALDILAENDEFRENEESCLAFRRASSVLKSLPFPVTSMKDTEGIPCLGDKVKRIIEEIIEDGESSEVKAVLNDERYKSFKLFTSVFGVGLKTAEKWFRMGFRTLEKIRSDKSLRFTQMQKAGFLYYEDLVSGVNRPEAEAVSTLVKEAVVTFLPDALVTMTGGFRLGHMTGHDVDFLITSPEATEDEEQQLLHKVTDFWKQQGLLLYCDILESTFEKEKRPSRGVDALDHFQKCFLILKLDHLRVHSAKSGQQEGKGWKAIRVDLVMCPYDRRAFALLGWTGSVQFNRDLRRYATHERKMMLDNHALYDKTKRVFLEAESEEEIFAHLGLDYIEPWERNA

SEQ ID NO: 14 - protein, 362 aa, Artificial Sequence
  M56-2=M56 without affinity tag and with N-terminal truncation
KISQYACQRRTTLNNYNELFTRALDILAENDEFRENEESCLAFRRASSVLKSLPFPVTSMKDTEGIPCLGDKVKRIIEEIIEDGESSEVKAVLNDERYKSFKLFTSVFGVGLKTAEKWFRMGFRTLEKIRSDKSLRFTQMQKAGFLYYEDLVSGVNRPEAEAVSTLVKEAVVTFLPDALVTMTGGFRLGHMTGHDVDFLITSPEATEDEEQQLLHKVTDFWKQQGLLLYCDILESTFEKEKRPSRGVDALDHFQKCFLILKLDHLRVHSAKSGQQEGKGWKAIRVDLVMCPYDRRAFALLGWTGSVQFNRDLRRYATHERKMMLDNHALYDKTKRVFLEAESEEEIFAHLGLDYIEPWERNA

SEQ ID NO: 15 - protein, 379 aa, Artificial Sequence
  M55-1=M55 without affinity tag
SPSPVPGSQNVPAPVVKKISQYACQRRTTLNNYNELFTRALDILAENDEFRENEESCLAFRRASSVLKSLPFPITSMKDTEGIPCLGDKVKRIIEEIIEDGESSEVKAVLNDERYKSFKLFTSVFGVGLKTAEKWFRMGFRTLERIRSDKSLRFTQMQKAGFLYYEDLVSGVNRPEAEAVSTLVKEAVVTFLPDALVTMTGGFRLGHQTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFKRDLRRYATHERKMMLDEHALYDKTKRVFLEAESEEEIFAHLGLDYIEPWERNA

SEQ ID NO: 16 - protein, 362 aa, Artificial Sequence
  M55-2=M55 without affinity tag and with N-terminal truncation
KISQYACQRRTTLNNYNELFTRALDILAENDEFRENEESCLAFRRASSVLKSLPFPITSMKDTEGIPCLGDKVKRIIEEIIEDGESSEVKAVLNDERYKSFKLFTSVFGVGLKTAEKWFRMGFRTLERIRSDKSLRFTQMQKAGFLYYEDLVSGVNRPEAEAVSTLVKEAVVTFLPDALVTMTGGFRLGHQTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFKRDLRRYATHERKMMLDEHALYDKTKRVFLEAESEEEIFAHLGLDYIEPWERNA